(19) United States
(12) Patent Application Publication
(10) Pub. No.: US 2023/0314359 A1
(43) Pub. Date: Oct. 5, 2023

(54) ELECTROCHEMICAL SENSOR

(71) Applicant: SUMITOMO CHEMICAL COMPANY, LIMITED, Tokyo (JP)

(72) Inventors: Masafumi YOKOYAMA, Hitachi-shi (JP); Masatomo SHIBATA, Hitachi-shi (JP)

(73) Assignee: SUMITOMO CHEMICAL COMPANY, LIMITED, Tokyo (JP)

(21) Appl. No.: 18/023,640

(22) PCT Filed: Jun. 3, 2021
(86) PCT No.: PCT/JP2021/021161

§ 371 Date: Feb. 27, 2023

(30) Foreign Application Priority Data
Aug. 31, 2020 (JP) 2020-145284

(51) Int. Cl. G01N 27/30; G01N 27/416

(52) U.S. Cl. CPC G01N 27/30; G01N 27/416

(57) ABSTRACT

There is provided an electrochemical sensor that electrochemically detects a specific substance in a test sample 50 by bringing a liquid test sample 50 into contact with sensor electrodes 20 arranged on a support 10, the electrochemical sensor including a holding structure 30 that constitutes a non-sealed finite space facing the sensor electrodes 20 and holds the test sample 50 that is brought into contact with the sensor electrodes 20 in the finite space.

### CROSS-REFERENCE TO RELATED APPLICATIONS

The present application claims priority under 35 U.S.C. § 371 to International Patent Application No. PCT/JP2021/021161, filed Jun. 3, 2021, which claims priority to and the benefit of Japanese Patent Application No. 2020-145284, filed on Aug. 31, 2020. The contents of these applications are hereby incorporated by reference in their entireties.

### TECHNICAL FIELD

The present disclosure relates to an electrochemical sensor that electrochemically detects a specific substance in a liquid test sample.

### DESCRIPTION OF RELATED ART

Some electrochemical sensors that electrochemically detect a specific substance in a liquid test sample are configured to perform detection by pouring a test sample, in addition to those used by immersing in the test sample stored in a container (see, for example, Patent document 1).

### PRIOR ART DOCUMENT

### Patent Document

[Patent Document 1] JP-A-2006-47125

### SUMMARY OF THE DISCLOSURE

### Problem to be Solved by the Disclosure

However, in the case of performing detection by pouring the test sample, there is a possibility that detection accuracy equivalent to that in the case of performing detection by immersing in the test sample cannot be obtained.

The present disclosure provides a technique capable of obtaining good detection accuracy even when detection is performed by pouring a test sample.

### Means for Solving the Problem

According to an aspect of the present disclosure, there is provided an electrochemical sensor that electrochemically detects a specific substance in a liquid test sample by bringing it into contact with sensor electrodes arranged on a support, the electrochemical sensor including:

a holding structure that constitutes a non-sealed finite space facing the sensor electrodes and holds the test sample that is brought into contact with the sensor electrodes in the finite space.

### Advantage of the Disclosure

According to the present disclosure, good detection accuracy equivalent to that obtained when the sensor electrodes is immersed in the test sample can be obtained, even when detecting by pouring the test sample over the sensor electrodes.

### BRIEF DESCRIPTION OF THE DRAWINGS

FIG. 1 is a perspective view illustrating a configuration example of a main part of an electrochemical sensor according to one aspect of the present disclosure.

FIG. 2 is a side cross-sectional view illustrating a configuration example of a main part of an electrochemical sensor according to one aspect of the present disclosure, and is a view illustrating a cross section taken along line A-A in FIG. 1.

FIG. 3 is an explanatory view schematically illustrating an example of a meniscus structure due to a surface tension of liquid.

FIGS. 4(a)-4(c) are explanatory views illustrating a specific example of a cyclic voltammogram corresponding to a measurement result by sensor electrodes 20 of the electrochemical sensor according to one aspect of the present disclosure, and for the same test sample, FIG. 4(a) is a view illustrating a cyclic voltammogram when the test sample is poured, FIG. 4(b) is a view illustrating a cyclic voltammogram when immersed in a test sample stored in a container, and FIG. 4(c) is a view illustrating a cyclic voltammogram when no test sample is held as a reference example.

FIG. 5 is an explanatory view illustrating a specific example of reproducibility of a measurement result by the sensor electrodes 20 of the electrochemical sensor according to one aspect of the present disclosure.

FIG. 6 is an explanatory view illustrating an example of the cyclic voltammogram corresponding to the measurement result by the sensor electrodes 20 of the electrochemical sensor, which differs depending on a holding amount of the test sample, according to one aspect of the present disclosure.

FIG. 7 is an explanatory view schematically illustrating an example of a size and a shape of the main part of the electrochemical sensor according to one aspect of the present disclosure.

FIG. 8 is a side cross-sectional view illustrating a configuration example of a main part of an electrochemical sensor according to another aspect of the present disclosure.

FIGS. 9(a)-9(h) are explanatory views (Part 1) illustrating a modified configuration example of the main part of the electrochemical sensor according to the present disclosure.

FIGS. 10(a)-10(h) are explanatory views (Part 2) illustrating a modified configuration example of the main part of the electrochemical sensor according to the present disclosure.

FIGS. 11(a)-11(e) are explanatory views (part 3) illustrating a modified configuration example of the main part of the electrochemical sensor according to the present disclosure.

FIGS. 12(a)-12(e) are explanatory views (part 4) illustrating a modified configuration example of the main part of the electrochemical sensor according to the present disclosure,

FIGS. 13(a)-13(d) are explanatory views (No. 5) illustrating a modified configuration example of the main part of the electrochemical sensor according to the present disclosure.

FIGS. 14(a)-14(g) are explanatory views (No. 6) illustrating a modified configuration example of the main part of the electrochemical sensor according to the present disclosure.

### DETAILED DESCRIPTION OF THE DISCLOSURE

### <One Aspect of the Present Disclosure>

First, a configuration example of an electrochemical sensor according to one aspect of the present disclosure will be described.

### (1) Configuration Example of an Electrochemical Sensor

An electrochemical sensor is a sensor that electrochemically detects a specific substance in a liquid test sample. In one aspect of the present disclosure, a case of detecting uric acid contained in urine collected from a subject will be described as an example. That is, in one aspect of the present disclosure, urine collected from a subject is exemplified as a liquid test sample, and uric acid contained in urine is exemplified as a specific substance to be detected. The concentration of the uric acid in urine is detected, for example, by electrolyzing a substance contained in the urine under specific conditions and utilizing an electrochemical reaction (e.g., oxidation-reduction reaction) that occurs at that time.

Further, in one aspect of the present disclosure, the electrochemical sensor is configured to detect the uric acid contained in urine by pouring urine, which is a test sample, over the electrochemical sensor. Thereby, a very simple examination becomes possible, and excellent convenience for the subject who uses the electrochemical sensor can be obtained, and the electrochemical sensor can detect the uric acid contained in urine not only by pouring urine as a test sample, but also by immersing in the urine held in a container.

An electrochemical sensor according to one aspect of the present disclosure is configured as described below in order to correspond to the usage aspect as described above.

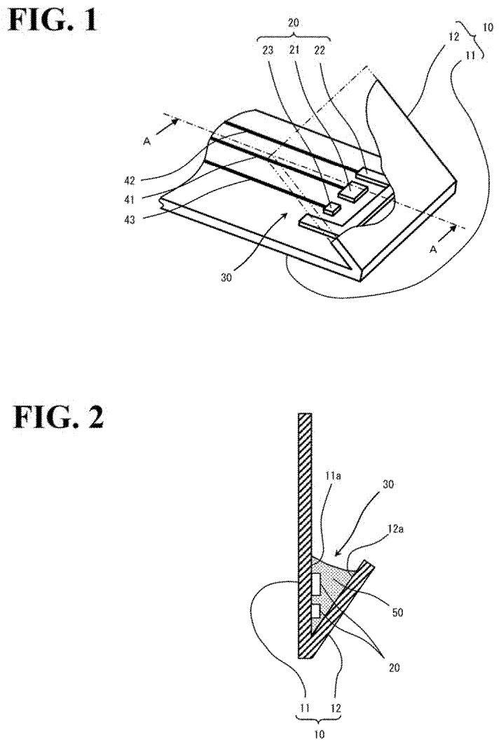

FIG. 1 is a perspective view illustrating a configuration example of a main part of an electrochemical sensor according to one aspect of the present disclosure. FIG. 2 is a side cross-sectional view illustrating A-A cross section in FIG. 1.

As illustrated in FIGS. 1 and 2, an electrochemical sensor according to one aspect of the present disclosure includes a support 10 and sensor electrodes 20.

### (Support)

The support 10 supports the sensor electrodes 20, and for example, has a base piece portion 11 comprising a strip-shaped plate-like member in plan view, and is configured so that the sensor electrodes 20 is arranged on a surface of the base piece portion 11 on one end side in a longitudinal direction. The other end in the longitudinal direction of the base piece portion 11 is configured to be connectable to a measuring device (not illustrated) such as a potentiostat that performs a predetermined voltage sweep operation for the sensor electrodes 20.

The base piece portion 11 comprises, for example, an insulating material having a mechanical strength that does not cause deformation or breakage when urine, which is a test sample, is poured over it. Specifically, the base piece portion 11 may comprise an insulating material such as, for example, an insulating resin material, ceramic, glass, plastic, a combustible material, a biodegradable material, non-woven fabric, or paper. As the base piece portion 11, a base material formed of, for example, polyethylene (PE), polyethylene terephthalate (PET), epoxy resin, etc., can be suitably used. Further, as the base piece portion 11, a semiconductor base material or a metal base material configured with the surface supporting the sensor electrodes 20 having insulating properties, can also be used.

Wires 41, 42 and 43 are provided on the surface of the base piece portion 11 on which the sensor electrodes 20 are arranged. The wires 41, 42, 43 correspond to a working electrode 21, a counter electrode 22, and a reference electrode 23 respectively, which will be described later, in the sensor electrodes 20, and are arranged so as to individually connect them to the measuring device. The wires 41, 42, 43 can be formed using a conductive metal material such as copper (Cu), aluminum (Al), gold (Au), and platinum (Pt). The wires 41, 42, 43 are covered with a resist, etc., that prevents the test sample (urine) from adhering to the wires 41, 42, 43.

A protruding piece portion 12 is provided on an arrangement side of the sensor electrodes 20 in the base piece portion 11 of the support 10. That is, the support 10 includes a protruding piece portion 12 in addition to the base piece portion 11. The protruding piece portion 12 constitutes a holding structure 30 whose details will be described later.

### (Sensor Electrode)

The sensor electrodes 20 include a working electrode 21, a counter electrode 22 and a reference electrode 23. The counter electrode 22 and the reference electrode 23 are provided near the working electrode 21. A wire 31 is connected to the working electrode 21, a wire 42 is connected to the counter electrode 22, and a wire 43 is connected to the reference electrode 23.

The working electrode 21 is configured to cause an oxidation-reduction reaction on the surface of the working electrode 21 when a predetermined voltage is applied in a state where urine, which is a test sample, exists between the working electrode 21 and the counter electrode 22. More specifically, when a predetermined voltage is applied between the working electrode 21 and the counter electrode 22 with the test sample (electrolyte) adhered thereto, the working electrode 21 is configured as a laminate of a diamond film and a support member (not illustrated). The diamond film (not illustrated) is a film that causes an oxidation-reduction reaction on its surface, which is the oxidation-reaction of a predetermined component (predetermined reactive species, e.g., uric acid) contained in the test sample, and the support member is a member that supports the diamond film. In this case, the working electrode 21 is arranged such that the supporting member is located on the side of the base piece portion 11. Thus, the electrochemical sensor including the working electrode 21 having a diamond film is also called a “diamond sensor”.

The diamond film constituting the working electrode 21 is a polycrystalline film. The diamond film may be a diamond-like carbon (DLC) film, a glassy carbon (GC) film, etc. The case of using the term “diamond film” in this specification, includes the case of referring to a polycrystalline diamond film, the case of referring to a DLC film, the case of referring to a GC film, and the case of referring to a combination thereof. The diamond film is preferably p-type. In order to form a p-type diamond film, the diamond film preferably contains an element such as boron (B) at a concentration of 1×10¹⁹ cm⁻³ or more and 1×10²² cm⁻³ or less. The B concentration in the diamond film can be measured, for example, by secondary ion mass spectroscopy (SIMS). The diamond film can grow (synthesize) by a chemical vapor deposition (CVD) method such as a hot filament CVD method, a plasma CVD method, or a physical vapor deposition (PVD method) such as an ion beam method or an ionization deposition method. etc. When the hot filament CVD method is used to grow the diamond film, a tungsten filament, for example, can be used as a filament. The thickness of the diamond film can be, for example, 0.5 μm or more and 10 μm or less, preferably 2 μm or more and 4 μm or less.

The support member that constitutes the working electrode 21 is formed using a material (different material) other than diamond. The support member preferably comprises an electrically conductive material. The support member preferably comprises, for example, silicon (Si) alone, a silicon compound, or a metal substrate. That is, the support member preferably comprises a silicon substrate or a metal substrate. Specifically, the support member preferably comprises any one of a single-crystal Si substrate, a polycrystalline Si substrate, a silicon carbide substrate (SiC substrate), and a metal substrate.

The counter electrode 22 is provided so as to surround the working electrode 21 and the reference electrode 23. As the counter electrode 22, an electrode comprising metal such as platinum (Pt), gold (Au), copper (Cu), palladium (Pd), nickel (Ni), silver (Ag), diamond electrode, boron-doped diamond A (BDD) electrode, a carbon electrode, etc., can be used. The counter electrode 22 can be formed by a known method such as a semi-additive method and a subtractive method. By applying a predetermined voltage between the working electrode 21 and the counter electrode 22, with the test sample adhered thereto, an oxidation-reduction reaction of a predetermined component (predetermined reactive species, such as uric acid) in the test sample occurs at the working electrode 21 and the counter electrode 22. Thereby, current flows between the working electrode 21 and the counter electrode 22. That is, the counter electrode 22 is an electrode for passing the current generated by the electrochemical reaction to the working electrode 21.

The reference electrode 23 is a reference electrode for determining a potential of the working electrode 21. As the reference electrode 23, for example, a silver/silver chloride (Ag/AgCl) electrode, etc., can be used. Further, as the reference electrode 23, a standard hydrogen electrode, a reversible hydrogen electrode, a palladium/hydrogen electrode, a saturated calomel electrode, a carbon electrode, a diamond electrode, a BDD electrode, etc., can be used. Further, as the reference electrode 23, an electrode comprising metal such as Pt, Au, Cu, Pd, Ni, Ag, etc., can be used. The reference electrode 23 can be formed by a known technique such as dispensing or screen printing.

### (Holding Structure)

The protruding piece portion 12 of the support 10 constitutes a holding structure 30. That is, the electrochemical sensor according to one aspect of the present disclosure includes the holding structure 30.

The holding structure 30 is arranged on one end side in a longitudinal direction of the base piece portion 11 (that is, on the side where the sensor electrodes 20 are arranged), and as illustrated in FIG. 2, holds the test sample 50 around the sensor electrodes 20, so that the test sample 50 supplied by pouring over the electrochemical sensor is maintained in contact with the sensor electrodes 20.

In order to hold the test sample 50 by the holding structure 30, a plate-shaped protruding piece portion 12 is arranged at the edge of the base piece portion 11, in such a manner as extending so as to fold back from the edge, and the protruding piece portion 12 is arranged to extend from one end side of the base piece portion 11 toward the other end side while increasing a distance from the base piece portion 11. That is, the base piece portion 11 and the protruding piece portion 12 are arranged so as to intersect with each other, and are configured to be integrated on the intersecting side. Thereby, the base piece portion 11 and the protruding piece portion 12 have a positional relationship in such manner as drawing a substantially V shape when viewed from the side.

The base piece portion 11 and the protruding piece portion 12, which are in a substantially V-shaped positional relationship, have two constituent surfaces 11a and 12a arranged to face each other. The term “opposing” as used herein includes the case where they are not parallel to each other as long as they are facing each other. One surface 11a of the two constituent surfaces is the surface of the base piece portion 11 on which the sensor electrodes 20 are attached. The other surface 12a of the two constituent surfaces is the surface of the protruding piece portion 12 on the base piece portion 11 side. These constituent surfaces 11a and 12a constitute a non-sealed finite space facing the sensor electrodes 20. That is, the holding structure 30 arranged on the side where the sensor electrodes 20 are arranged, constitutes a non-sealed finite space facing the sensor electrodes 20. Specifically, the holding structure 30 is sandwiched between the two constituent surfaces 11a and 12a, and constitutes a non-sealed finite space, with the protruding piece portion 12 opened at the tip side, and further, lateral sides of the two constituent surfaces 11a and 12a opened.

The non-sealed finite space is formed for holding the test sample 50. Such a finite space is adapted to hold the test sample 50 even when it is non-sealed, by utilizing a surface tension of the liquid test sample 50. That is, the holding structure 30 is configured to hold the test sample 50 by utilizing the surface tension of the liquid test sample 50. A specific mode of holding the test sample 50 by the holding structure 30 will be described later in detail.

The protruding piece portion 12 for constituting such a holding structure 30 can be formed by bending one end side of the base piece portion 11. In that case, the bending may be performed at the time of manufacturing the electrochemical sensor, or may be performed by the subject immediately before using the electrochemical sensor. However, the protruding piece portion 12 does not necessarily have to be formed integrally with the base piece portion 11, but may be formed separately from the base piece portion 11 and can be attached to the base piece portion 11. In the case of a separate body from the base piece portion 11, the protruding piece portion 12 may be configured to be detachable from the base piece portion 11. That is, the holding structure 30 may be detachable from the support 10 that supports the sensor electrodes 20.

The protruding piece portion 12 can comprise the same material as the base piece portion 11, regardless of whether it is integral or separate. When the same material is used, the process of manufacturing the electrochemical sensor can be simplified, the procurement of materials can be facilitated, and the cost can be reduced accordingly. However, the protruding piece portion 12 doesn't necessarily comprise the same material, and may comprise a material different from that of the base piece portion 11. When comprising a different material, it becomes possible to make the support 10 and the holding structure 30 have different functions (roles).

Specifically, as a material for forming the protruding piece portion 12, for example, any of insulating materials such as insulating resin materials, ceramics, glass, plastics, combustible materials, biodegradable materials, non-woven fabrics, paper, etc., or an appropriate combination thereof can be used. Particularly, for example, PE, PET, epoxy resin, etc. can be preferably used.

### (2) Example of a Processing Operation of the Electrochemical Sensor

Next, as an example of a processing operation of the electrochemical sensor according to one aspect of the present disclosure, a procedure for detecting a uric acid concentration by an electrochemical measurement, will be described. Here, a case in which the test sample 50 is a uric acid solution, which is a test liquid and the concentration of the uric acid solution is detected, will be taken as an example. As the test liquid, 50 mg of the uric acid was added to 100 cc of pH 7 phosphate buffer solution, stirred, and dissolved until solids disappeared.

The procedure for detecting the uric acid concentration using the electrochemical sensor includes: a procedure for connecting the support 10 of the electrochemical sensor to a measuring device (potentiostat, etc.) not illustrated (step 1), a procedure for supplying (contacting) the test sample 50 to the sensor electrodes 20 (step 2), and a procedure for measuring a current value flowing through the oxidation-reduction reaction of the uric acid, by applying a voltage between the working electrode 21 and the counter electrode 22 of the sensor electrodes 20 while the test sample 50 is in contact, thereby causing an oxidation-reduction reaction of uric acid on the surface of a diamond film of the working electrode 21 (step 3), a procedure for measuring a potential difference (voltage difference) between the working electrode 21 and the reference electrode 23 while the test sample 50 is in contact (step 4), and a procedure for quantifying the uric acid concentration based on the measured current value and potential difference (step 5).

### (Step 1)

In this step, the electrochemical sensor is connected to the measuring device. Specifically, each of the wires 41, 42, 43 in the support 10 of the electrochemical sensor and the measuring device (potentiostat, etc.) is electrically connected. The measuring device is configured to be able to perform a predetermined voltage sweep operation for the sensor electrodes 20, and for example, it has a voltage application unit, a current measurement unit, a potential difference measurement unit, and a potential adjustment unit. The voltage application unit is configured to apply a voltage between the working electrode 21 and the counter electrode 22 when a predetermined circuit is formed by connecting the wires 41, 42 and 43. The current measurement unit is configured to measure a current generated by the oxidation-reduction reaction of uric acid. The potential adjustment unit is configured to measure a potential difference between the working electrode 21 and the reference electrode 23. The potential adjustment unit is configured to keep the potential of the working electrode 21 constant with the potential of the reference electrode 23 as a reference, based on the potential difference measured by the potential difference measurement unit.

### (Step 2)

After connecting the electrochemical sensor and the measuring device (potentiostat, etc), for example, the test sample 50 is poured over the electrochemical sensor. Thereby, the test sample 50 is held by the holding structure 30, and the test sample 50 reaches and adheres to the surfaces of the sensor electrodes 20. A specific mode of holding the test sample 50 by the holding structure 30 at this time will be described later in detail.

### (Step 3)

By applying a predetermined voltage between the working electrode 21 and the counter electrode 22 by the voltage application unit of the measurement mechanism in a state where the test sample 50 is adhered to the surfaces of the sensor electrodes 20, the oxidation-reduction reaction of the uric acid occurs on the surface of the diamond film of the working electrode 21. A current (reaction current) flows through the working electrode 21 due to the oxidation-reduction reaction of the uric acid. The value of this reaction current is measured by, for example, cyclic voltammetry using a measurement mechanism. For example, cyclic voltammetry conditions are as follows. Voltage range: range including 0 V or more and 1 V or less, sweep speed: 0.1 V/s or more and 1 V/s or less. The value of a reaction current may be measured using techniques such as square wave voltammetry (rectangular wave voltammetry), differential pulse voltammetry, normal pulse voltammetry, and alternating current voltammetry.

### (Step 4)

The potential difference between the working electrode 21 and the reference electrode 23 is measured by the potential difference measurement unit of the measuring mechanism, with the test sample in contact with the surfaces of the sensor electrodes 20.

### (Step 5)

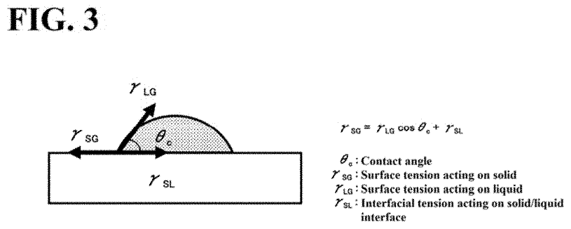

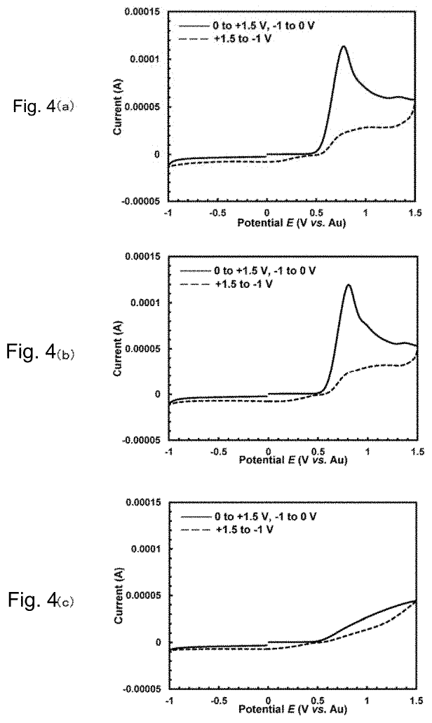

For example, a cyclic voltammogram is created from the value of the reaction current measured in step 3, and a current value at an oxidation peak is obtained. Based on the acquired oxidation peak current value and the value of the potential difference measured in step 4, the uric acid concentration in the test sample is calculated (quantified). It is disclosed in a known document (for example, Anal. Methods, 2018.10, 991-996, see FIGS. 3 and 4) that the value of the reaction current is correlated with the uric acid concentration in the test sample. Accordingly, when the relationship between the reaction current value and the uric acid concentration is determined in advance, the uric acid concentration can be quantified based on the measured reaction current value.

### (3) Holding Mode of the Test Sample

Next, a specific mode of holding the test sample 50 by the holding structure 30 in the series of processing operations described above, will be described.

### (Surface Tension)

When the subject pours the test sample 50 over the electrochemical sensor, the test sample 50 is supplied to the holding structure 30. The holding structure 30 to which the test sample 50 is supplied constitutes a non-sealed finite space facing the sensor electrodes 20. More specifically, the holding structure 30 has two constituent surfaces 11a and 12a, and by arranging these two constituent surfaces 11a and 12a facing each other, the non-sealed finite space is formed.

On the other hand, since the test sample 50 supplied to the holding structure 30 is liquid, surface tension acts on it as illustrated in FIG. 3. That is, in the finite space, the liquid test sample 50 may have a curved surface due to surface tension rather than a flat surface. This is hereinafter also referred to as a meniscus structure.

Accordingly, even when the holding structure 30 constitutes the non-sealed finite space, a certain amount of the test sample 50 is held by the holding structure 30 by forming the test sample 50 into a meniscus structure in the non-sealed portion due to surface tension. That is, when the test sample 50 is poured over the electrochemical sensor, the holding structure 30 holds a test sample 50 that is brought into contact with the sensor electrodes 20 in the non-sealed finite space. Thereby, the finite space positioned around the sensor electrodes 20 is filled with the test sample 50, and an immersion state of the sensor electrodes 20 in the filled test sample 50 is maintained.

In this manner, the holding structure 30 holds the test sample 50, utilizing the surface tension of the test sample 50. Therefore, the holding structure 30 can reliably hold a necessary and sufficient constant amount of the test sample 50, with a very simple configuration.

### (Verification of a Measurement Result)

When the holding structure 30 holds the test sample 50, the periphery of the sensor electrodes 20 is filled with the test sample 50 held by the holding structure 30. Thereby, only the test sample 50 comes into contact with the exposed surfaces of the sensor electrodes 20 excluding the surface supported by the support 10. That is, the holding structure 30 holds the test sample 50 so that only the test sample 50 contacts the exposed surfaces of the sensor electrodes 20 and the air, other members, etc. do not contact the sensor electrodes.

When the periphery of the sensor electrodes 20 is filled with the test sample 50 and only the test sample 50 is in contact with the exposed surfaces of the sensor electrodes 20, the sensor electrodes 20 can be in a state substantially equivalent to a state of being immersed in the test sample 50 stored in a container. Accordingly, even when the test sample 50 is supplied to the sensor electrodes 20 by pouring, a measurement result with good accuracy equivalent to that of immersion in the test sample 50 stored in the container, can be obtained.

FIGS. 4(a)-4(c) are explanatory views illustrating a specific example of a cyclic voltammogram corresponding to the measurement result by the sensor electrodes 20. Regarding the same test liquid as the test sample 50, FIG. 4(a) is a view illustrating a cyclic voltammogram when the test liquid is poured, FIG. 4(b) is a view illustrating a cyclic voltammogram when immersed in the test liquid stored in the container, and (c) is a view illustrating a cyclic voltammogram when no test liquid is held as a reference example. Each cyclic voltammogram was obtained under the same condition except for a supply mode of the test liquid.

An oxidation peak current value in the cyclic voltammogram illustrated in FIG. 4 (a) is approximately equal to an oxidation peak current value in the cyclic voltammogram illustrated in FIG. 4 (b). That is, according to the measurement result illustrated in FIGS. 4 (a) and 4 (b), even when the test sample 50 is poured over the sensor electrodes 20, it is found that a measurement result with good accuracy equivalent to that obtained when the sensor electrodes 20 is immersed in the test sample 50 stored in a container, can be obtained.

Specifically, regarding the test sample 50, which is the same test liquid, a difference (A−B or B−A) between the intensity B of a detection signal obtained by immersing the sensor electrodes 20 in the test sample 50 stored in a container with respect to the intensity A of a detection signal obtained by the sensor electrodes 20 while the holding structure 30 holds the test sample 50, is 10% or less of the intensity A (ie (A−B or B−A)/A≤10%). Thus, when the difference between the intensity B and the intensity A is 10% or less, it can be said that the measurement result is that the intensity A and the intensity B can be obtained with the same degree of good accuracy.

Accordingly, based on such a measurement result, as for the detection result of the uric acid concentration in the test sample 50, good detection accuracy can be obtained even when performing detection by pouring the test sample 50.

Incidentally, as illustrated in FIG. 4 (c), when the test sample 50 is not held, the contact state of the test sample 50 with the sensor electrodes 20, cannot be maintained. Therefore, an oxidation peak current value cannot be obtained in the cyclic voltammogram. This means that the reason why good detection accuracy equivalent to that in the immersed state can be obtained even when detection is performed by pouring the test sample 50 is that the holding structure 30 holds the test sample 50.

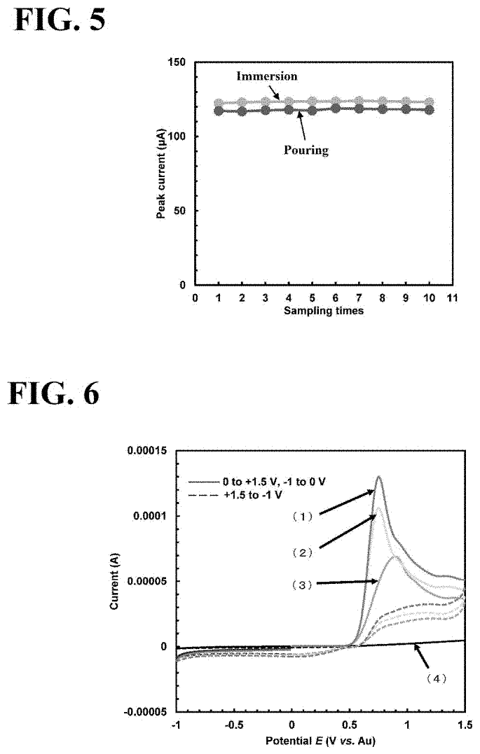

Here, one cyclic voltammogram is illustrated as a measurement result by the sensor electrodes 20. However, it has already been confirmed that equivalent measurement results can be obtained even when the test sample 50, which is the same test liquid, is repeatedly measured a plurality of times. FIG. 5 is an explanatory view illustrating a specific example of reproducibility of the measurement result by the sensor electrodes 20. The figure shows the oxidation peak current value in the cyclic voltammogram obtained by repeating measurement for the test sample 50 which is the same test liquid, for example, 10 times. According to the illustration, it is found that there is no difference other than an error component in each measurement result, and each variation is suppressed to about 1% to 2%, not only when the test sample 50 is poured over the sensor electrodes 20, but also when the sensor electrodes 20 is immersed in the test sample 50. That is, the measurement result obtained by the sensor electrodes 20 have reproducibility even when the measurement is repeated a plurality of times.

### (Holding Amount of the Test Sample)

In order to obtain the above measurement result, the holding structure 30 needs to hold a necessary and sufficient amount of the test sample 50, that is, the amount of the test sample 50 that fills the periphery of the sensor electrodes 20. Here, the holding amount of the test sample 50 by the holding structure 30 will be described.

FIG. 6 is an explanatory view illustrating an example of how the cyclic voltammogram corresponding to the measurement result by the sensor electrodes 20 differs depending on the holding amount of the test sample 50. In the figure, (1) is a cyclic voltammogram obtained in a state where the holding structure 30 holds the test sample 50 in an amount reaching the tip of the protruding piece portion 12 (that is, in a state of total liquid immersion), and (2) is a cyclic voltammogram obtained in a state where the holding structure 30 holds the test sample 50 in an amount reaching near an upper end of the sensor electrodes 20 (that is, in a state of total liquid immersion with less liquid). (3) is a cyclic voltammogram obtained in a state where only the working electrode 21 and the counter electrode 22 of the sensor electrodes 20 are immersed in liquid. (4) is a cyclic voltammogram obtained in a state where the sensor electrodes 20 are substantially not immersed in liquid. Each cyclic voltammogram is obtained for the test sample 50, which is the same test liquid, under the same condition except for the holding amount.

According to each cyclic voltammogram (1) to (4) illustrated in FIG. 6, it is found that the oxidation peak current value (that is, the intensity of the detection signal obtained by the sensor electrodes 20) increases as the holding amount of the test sample 50 increases. Specifically, when the test sample 50 reaches the tip of the protruding piece 12 and an entire sensor electrodes 20 is completely immersed in the liquid (see (1) in FIG. 6), a best measurement result is obtained. However, when at least the working electrode 21 and the counter electrode 22 of the sensor electrodes 20 are immersed in liquid, it is found that the oxidation peak current value in the cyclic voltammogram is obtained (see (3) in FIG. 6). That is, in order to detect the uric acid concentration in the test sample 50, the holding structure 30 may hold the test sample 50 in such a manner that at least an entire exposed surface of the working electrode 21 is in contact with the test sample 50. The holding amount of the test sample 50 held in this manner corresponds to a lower limit value of the holding amount of the test sample 50 held by the holding structure 30. Then, in order to obtain a better measurement result, it is desirable that the holding structure 30 holds the test sample 50 in such a manner that an entire sensor electrodes 20 is completely immersed in the liquid. The holding amount of the test sample 50 held in this manner corresponds to a preferable value of the holding amount of the test sample 50 held by the holding structure 30.

When the holding amount of the test sample 50 is a preferable value, at least the working electrode 21 of the sensor electrodes 20 is not only in contact with the test sample 50 at its exposed surface, but is filled with a sufficient amount of the test sample 50 around the exposed surface.

In other words, the holding structure 30 holds the test sample 50, which is the test sample 50, in a region having an area larger than an area of the working electrode 21 when viewed from above. When the test sample 50 is held in a wider area than the working electrode 21, the surface of the working electrode 21 is completely in contact with the test sample 50, which is very suitable for obtaining a good measurement result.

Further, the holding structure 30 holds the test sample, which is the test sample 50, in a manner of maintaining a sufficient thickness on the exposed surface of the working electrode 21. Specifically, the test sample 50 is held in such a manner that the exposed surface of the working electrode 21 maintains a thickness equal to or greater than a diffusion length of a component to be detected. The component to be detected is a predetermined component (predetermined reactive species such as uric acid) in the test sample 50, and the diffusion length of the component to be detected is a length (distance) from the exposed electrode surface at which the diffusion of the component occurs. Thus, when the test sample 50 is held with a thickness equal to or greater than the diffusion length of the component to be detected with respect to the working electrode 21, a necessary and sufficient holding amount of the test sample can be secured over an entire exposed surface of the working electrode 21, which is very suitable for obtaining a good measurement result.

The holding amount of the test sample 50 as described above has a correlation with a volume of the non-sealed finite space in the holding structure 30. That is, a size and a shape of the finite space in the holding structure 30 are formed so as to be able to hold the test sample 50 utilizing the surface tension of the test sample 50, and so as to be able to hold an amount of the test sample 50 that fills the periphery of the sensor electrodes 20.

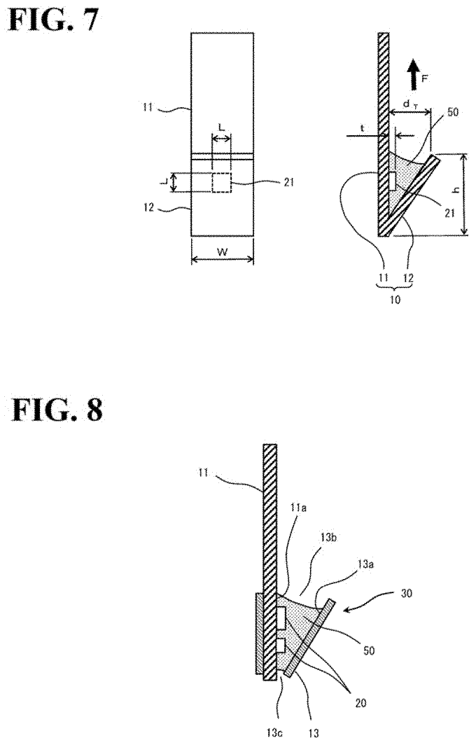

The size and the shape of the finite space in the holding structure 30 are configured as illustrated in FIG. 7 according to one aspect of the present disclosure.

FIG. 7 is an explanatory view schematically illustrating an example of the size and the shape of the main part of the electrochemical sensor according to one aspect of the present disclosure. In the figure, only the working electrode 21 of the sensor electrodes 20 is illustrated, and the illustration of the other electrode is omitted.

For example, a case where the working electrode 21 of the sensor electrodes 20 has a sensor width L of 1.8 mm and a sensor thickness t of 0.4 mm, is considered. In this case, the finite space in the holding structure 30 is capable of holding the test sample 50 in a holding amount that is a preferable value described above, when a formation width W is about 6 mm, a rising height h of the protruding piece portion 12 is about 5 mm to 10 mm, and an opening length d_{T} on the tip side of the protruding piece portion 12 is about 5 mm to 7 mm. When configured with such a size and shape, the holding structure 30 has a holding capacity of the test sample 50 of, for example, 0.075 ml or more and 0.30 ml or less. That is, when the holding capacity of the test sample 50 is 0.075 ml or more and 0.30 ml or less, the holding structure 30 can hold the test sample 50 in a suitable holding amount. Thereby, a state where an entire sensor electrodes 20 is completely immersed in the liquid, can be reproduced.

However, the uric acid concentration in the test sample 50 can be detected even when the holding amount of the test sample 50 held by the holding structure 30 is not the preferable value but the lower limit value described above. The holding capacity of the test sample 50 in the holding structure 30 at a lower limit is, for example, 0.01 ml or more. When the holding capacity of the test sample 50 is 0.01 ml or more and 0.30 ml or less, the holding structure 30 can bring at least the entire exposed surface of the working electrode 21 into contact with the test sample 50.

In this way, the size and the shape of the finite space in the holding structure 30 is formed so that the holding capacity of the test sample 50 is 0.01 ml or more and 0.30 ml or less, preferably 0.075 ml or more and 0.30 ml or less. With such a holding capacity, an immersion state of the sensor electrodes 20 in the test sample 50 is reproduced, and the uric acid concentration in the test sample 50 can be reliably detected. As for an upper limit, 0.30 ml or less may be sufficient because it is not useful to hold an amount exceeding a necessary and sufficient amount.

Regarding the holding amount of the test sample 50 by the holding structure 30 as described above, by using the finite space that constitutes the holding structure 30 and the surface tension of the test sample 50, which is the test sample 50, very high reproducibility can be obtained. For example, when the holding amount of the test sample 50 held by the holding structure 30 is repeatedly measured a plurality of times (for example, 10 times), the inventor of the present application has already confirmed that the variation in the measured value is very small (e.g. less than 10%). Further, even when a posture of the electrochemical sensor is changed (for example, tilted) after holding the test sample 50 by the holding structure 30, the inventor of the present application has already confirmed that the holding amount of the test sample 50 held by the holding structure 30 does not change significantly.

Further, regarding the holding amount of the test sample 50 held by the holding structure 30, no significant change occurs as long as no external force is applied, at least until a time sufficient for measurement by the sensor electrodes 20 elapses. The necessary and sufficient time for measurement by the sensor electrodes 20 is, for example, about one minute. That is, the holding structure 30 is configured to maintain a holding state of the test sample 50, which is the test sample 50, for at least 1 minute when no external force is applied. Maintaining the holding state of the test sample 50 for at least 1 minute can suppress an adverse effect on the measurement by the sensor electrodes 20, and therefore detection of the uric acid concentration in the test sample 50 can be performed appropriately. The time required and sufficient for measurement by the sensor electrodes 20 is not necessarily limited to about 1 minute, and is appropriately specified according to a specification of the sensor electrodes 20 and a measuring device (potentiostat, etc.).

More specifically, regarding the holding amount of the test sample 50 held by the holding structure 30, no variation occurs in the holding amount of the test sample 50, or the variation in the volume that occurs is less than an average value±10%, preferably less than an average value±5%, even when there is a change in the posture of the support 10 or a change in an environmental condition within a predetermined range. Here, changing the posture of the support 10 within a predetermined range means, for example, rotating the sensor electrodes 20 from a horizontal state to a vertical state at a speed that does not cause a separation centrifugal force. Further, a change in an environmental condition within a predetermined range means, for example, a change in an environmental temperature from 0° C. to +30° C., a change in an air pressure from 960 hPa to 1060 hPa, and the like. Even when there is such a change in the posture or the environmental condition, there is no change in the holding amount of the test sample 50 held by the holding structure 30 at least until the time necessary and sufficient for measurement by the sensor electrodes 20 elapses, or when an amount of change is small, it is possible to ensure good test accuracy in detecting the uric acid concentration in the test sample 50.

### (Contact Angle of the Test Sample)

In order to hold the test sample 50 in the amount described above, the holding structure 30 utilizes the surface tension of the test sample 50 as described above. It can be considered as follows whether or not the surface tension of the test sample 50 can be utilized.

When holding the test sample 50 utilizing its surface tension, the force F (see FIG. 7) with which the test sample 50 tries to escape from the finite space of the holding structure 30 becomes “0”. In one aspect of the present disclosure, the force F is represented by formula (1) below.

F=2(d_{T}+W)×γ_{LG} cos θ_{C}−mg=2(d_{T}+W)×γ_{LG} cos θ_{C}−(d_{T}+d_{B})/2×Whρg=0 (1)

In formula (1), F is a magnitude of a detachment force, γ_{LG} is a surface tension acting on the test sample, θ_{C} is a contact angle, d_{T} is an upper opening length of the finite space, and d_{B} is AN opening length of a lower finite space (in one aspect of the present disclosure, d_{B}=0, W is a formation width of the finite space, h is a formation height of the finite space, m is a mass of the test sample, g is a gravitational acceleration, and ρ is a density of the test sample).

Formula (1) can be replaced by formula (2) below.

h=4(d_{T}+W)γ_{LG} cos θ_{C}/(d_{T}+d_{B})Wρg (2)

In formula (2), in the case of h>0, the holding structure 30 can hold some amount of the test sample 50 even when it is configured by a non-sealed finite space. For satisfying h>0, at least cos θ>0 needs to be satisfied. In other words, it can be said that the holding structure 30 can hold the test sample 50 as long as the contact angle θ_{C} is in a range of cos θC>0. The range of cos θ_{C}>0 is satisfied when the contact angle θ_{C} is 0° (deg) or more and less than 90° (deg).

That is, in the holding structure 30, constituent surfaces 11a and 12a of the finite space are configured respectively, such that the contact angle θ_{C} of the test sample 50 when the test sample 50 is brought into contact, is 0° or more and less than 90°. Thus, when the contact angle θ_{C} is 0° or more and less than 90°, the holding structure 30 can reliably utilize the surface tension of the test sample 50.

### (Detachment of the Test Sample)

When the measurement result by the sensor electrodes 20 is obtained with the test sample 50 held by the holding structure 30, thereafter, the non-sealed portion of the finite space that constitutes the holding structure 30 may be used to detach the test sample 50 in a held state from the holding structure 30. Specifically, by applying an external force to the holding structure 30 holding the test sample 50, the test sample 50 can be detached from the holding structure 30. The external force in that case includes, for example, a centrifugal force generated by manually holding and shaking the support 10. However, the external force is not limited to the centrifugal force, and may be, for example, a water absorbing power of a water absorbing paper member, cloth member, etc.

That is, the holding structure 30 is configured to detach the test sample 50 in the held state from the holding structure 30 (more specifically, from the non-sealed portion of the finite space) by applying the external force. Thereby, the electrochemical sensor according to one aspect of the present disclosure can detach the test sample 50 simply by applying the external force after use, even when the holding structure 30 is configured to hold the test sample 50. Therefore, convenience can be improved when discarding a used sensor.

### (4) Other Function of the Holding Structure

The holding structure 30 may also have functions described below in addition to the function of holding the test sample 50.

### (Specific Examples of Other Functions)

The holding structure 30 may also have a protective function to prevent an object other than the test sample 50 from contacting the sensor electrodes 20. Specifically, for example, when the rising height h of the protruding piece portion 12 that constitutes the holding structure 30 is about 5 mm to 10 mm, and the opening length d_{T} on the tip end side of the protruding piece portion 12 is about 5 mm to 7 mm, the sensor electrodes 20 are sufficiently covered by the protruding piece portion 12. Accordingly, a protective function of the sensor electrodes 20 is exhibited by the protruding piece portion 12, and for example, it is possible to prevent a user's hand from accidentally touching the sensor electrodes 20, which is very useful for preventing damage to the sensor electrodes 20 and ensuring inspection accuracy.

Further, the holding structure 30 may also have an evaporation prevention function to prevent the test sample 50 in the held state from evaporating. Specifically, by covering most of the finite space constituting the holding structure 30 with the constituent surfaces 11a and 12a and limiting the non-sealed portion to only a part, the test sample 50 can be prevented from evaporating. By reducing the area of the test sample 50 exposed to the outside air in this way, the test sample 50 can be prevented from evaporating. This is extremely useful in suppressing the change in the holding amount of the test sample 50 held by the holding structure 30.

### (Material for Forming the Holding Structure)

Incidentally, two constituent surfaces 11a and 12a that constitute the finite space in the holding structure portion 30, more specifically, the base piece portion 11 and the protruding piece portion 12 having the constituent surfaces 11a and 12a, comprise a material such as PE, PET, epoxy resin, etc., as described above. However, the following forming materials are preferable from a viewpoint of the function of the holding structure 30.

The base piece portion 11 and the protruding piece portion 12 preferably comprise an insulating material. When comprising the insulating material, it will not adversely and electrically affect a detection signal obtained by the sensor electrodes 20, even when the holding structure 30 holds the test sample 50 and causes the sensor electrodes 20 to detect uric acid, which is a specific substance contained in the test sample 50.

Further, at least the protruding piece portion 12 in the base piece portion 11 and the protruding piece portion 12, preferably comprise a light-transmitting material. This is because the state in which the holding structure 30 holds the test sample 50 can be visually recognized from outside when the holding structure 30 is light-transmitting.

Further, the base piece portion 11 and the protruding piece portion 12 preferably comprise a material that does not impregnate the test sample 50. This is because when the test sample 50 is not impregnated, the holding structure 30 will not be adversely affected by the impregnation of the test sample 50 (for example, fluctuation in the holding amount of the test sample 50).

Further, at least the protruding piece portion 12 in the base piece portion 11 and the protruding piece portion 12, preferably comprises a flexible material. This is because when it has flexibility, for example, even when the protruding piece portion 12 hits something, the protruding piece portion 12 is deformed so as to bend, thereby preventing damage to the holding structure 30, which improves convenience.

Further, the base piece portion 11 and the protruding piece portion 12 preferably comprise a material that does not contain a component that elutes into the test sample 50. This is because when the test sample 50 does not contain a component that elutes into the test sample 50, the holding structure 30 holds the test sample 50, and even when the sensor electrodes 20 detect the uric acid contained in the test sample 50, the detection signal obtained by the sensor electrodes 20 is not adversely affected (for example, detection of the eluted component).

Further, the base piece portion 11 and the protruding piece portion 12 preferably comprise a hydrophilic material. This is because the holding structure 30 can be easily filled with the test sample 50 when it is hydrophilic.

Further, the base piece portion 11 and the protruding piece portion 12 can comprise, for example, a material having a smooth surface at a contact portion with the test sample 50. When it has a smooth surface, it becomes possible to facilitate a transition to a holding state of the test sample 50 (inflow of the test sample 50). The smooth surface means a surface that is not subjected to uneven processing, roughening treatment, etc. However, it does not necessarily have a smooth surface, and for example, the contact portion with the test sample 50 may comprise a material having a rough surface. When it has a rough surface, it is expected that the test sample 50 will stay more easily. The rough surface refers to a surface on which some kind of roughening treatment, etc., is applied to a material constituting the surface.

### (5) Effect

According to the present aspect, one or more of the following effects can be obtained.
(a) The holding structure 30 that constitutes the non-sealed finite space facing the sensor electrodes 20 holds the test sample 50 that is brought into contact with the sensor electrodes 20 in the finite space. That is, when the test sample 50 is supplied to the sensor electrodes 20, the periphery of the sensor electrodes 20 is filled with the test sample 50 by holding the test sample 50 by the holding structure 30, and an immersion state of the sensor electrodes 20 in the filled test sample 50 is maintained. Accordingly, even when the test sample 50 is poured over the sensor electrodes 20, good detection accuracy equivalent to that obtained when the sensor electrodes 20 are immersed in the test sample 50, can be obtained.
(b) The holding structure 30 holds the test sample 50 utilizing the surface tension of the test sample 50. By utilizing the surface tension of the test sample 50 in this manner, a necessary and sufficient amount of the test sample 50 can be reliably held with a simple configuration.

Particularly, when the holding structure 30 has at least two constituent surfaces 11a and 12a and is configured to hold the test sample 50 between the constituent surfaces 11a and 12a, the test sample can be held with a very simple configuration.
(c) The holding structure 30 is configured so that the holding capacity of the test sample 50 is 0.01 ml or more and 0.30 ml or less, preferably 0.075 ml or more and 0.30 ml or less. With such a holding capacity, the state in which the sensor electrodes 20 are immersed in the test sample 50 can be reproduced, and a specific substance in the test sample 50 can be reliably detected.
(d) The holding structure 30 does not cause a variation in the holding amount of the test sample 50 even when a posture of the support 10 or an environmental condition changes within a predetermined range, or it is configured so that the variation in the capacity that occurs is less than the average value±10%. Accordingly, even when there is such a change in the posture or environmental condition, good inspection accuracy can be ensured for detection of the specific substance in the test sample 50, as long as there is no change in the holding amount of the test sample 50 held by the holding structure 30 or when the change amount is small at least until elapse of the time necessary and sufficient for measurement by the sensor electrodes 20.
(e) The holding structure 30 is configured such that the test sample 50 in a held state is detached from the holding structure 30 by applying an external force. Accordingly, even when the holding structure 30 is configured to hold the test sample 50, the test sample 50 can be detached simply by applying an external force after use. Therefore, convenience can be improved when discarding a used sensor.
(f) When the holding structure 30 also has a protective function to prevent objects other than the test sample 50 from coming into contact with the sensor electrodes 20, for example, a situation such as the user's hand accidentally touching the sensor electrodes 20 can be prevented. This is extremely useful for preventing damage to the sensor electrodes 20 and ensuring inspection accuracy.
(g) When the sensor electrodes 20 have the working electrode 21 and the counter electrode 22, and the working electrode 21 is composed of a diamond film, a specific substance in the test sample 50 can be electrochemically detected by utilizing the oxidation-reduction reaction caused by the diamond film. Accordingly, it is very useful for good detection of the specific substance in the test sample 50.
(h) When the sensor electrodes 20 have the working electrode 21 and the counter electrode 22, the holding structure 30 may hold the test sample 50 in such a manner that at least the entire exposed surface of the working electrode 21 is in contact with the test sample 50. When at least the entire exposed surface of the working electrode 21 is in contact with the test sample 50, the specific substance in the test sample 50 can be reliably detected.
(i) When the holding structure 30 comprises a light-transmitting material, the state in which the holding structure 30 holds the test sample 50 can be visually recognized from the outside, thereby improving convenience for a user.

### Another Aspect of the Present Disclosure

Next, an electrochemical sensor according to another aspect of the present disclosure will be described. Here, differences from the one aspect described above will be mainly described.

FIG. 8 is a side cross-sectional view illustrating a configuration example of a main part of an electrochemical sensor according to another aspect of the present disclosure.

As illustrated in FIG. 8, the electrochemical sensor according to another aspect of the present disclosure is configured such that a cap portion 13 is attached to a side of the support 10 including the base piece portion 11 on which the sensor electrodes 20 are arranged.

The cap portion 13 is formed in a cylindrical shape having a configuration surface 13a that is inclined with respect to the base piece portion 11. Due to the inclination of the configuration surface 13a, the cap portion 13 has an opening 13b on one end side (upper side in the drawing) of the cylindrical shape that is larger (larger area) than an opening 13c on the other end side (lower side in the drawing) of the cylindrical shape.

Then, the cap portion 13 is attached in the vicinity of an arrangement location of the sensor electrodes 20 on the base piece portion 11, with the base piece portion 11 inserted into the cylinder.

Attachment of the cap portion 13 is performed by, for example, fitting to the base piece portion 11. However, it is not necessarily limited to fitting, and for example, adhesion or sticking may be acceptable. In either case, it is preferable that the cap portion 13 is detachable.

Such a cap portion 13 may comprise the same material as the protruding piece portion 12 in one aspect of the present disclosure described above.

When the cap portion 13 is attached, two wall surfaces 11a and 13a are arranged in the cylinder of the cap portion 13 so as to face each other. One surface 11a out of the two wall surfaces is the surface of the base piece portion 11 on which the sensor electrodes 20 are attached. The other wall surface 13a of the two is the inclined surface 13a of the cap portion 13. Further, in addition to these wall surfaces 11a and 13a, two wall surfaces (not illustrated) connecting the wall surfaces 11a and 13a on the sides of the wall surfaces 11a and 13a are arranged in the cylinder of the cap portion 13. The four wall surfaces including each of the wall surfaces 11a and 13a are arranged to surround the sensor electrodes 20, and a non-sealed finite space surrounding the sensor electrodes 20 is configured. Specifically, an inside of the cylinder of the cap portion 13 is surrounded by four wall surfaces including the wall surfaces 11a and 13a, and the non-sealed finite space is configured, with the opening portions 13b and 13c opened.

The non-sealed finite space is formed to hold the test sample 50. Such a finite space holds the test sample 50 by utilizing the surface tension of the liquid test sample 50 even when it is not sealed. That is, the cap portion 13 is attached to the base piece portion 11 of the support 10 to constitute the non-sealed finite space facing the sensor electrodes 20, and functions as the holding structure 30 that holds the test sample 50 in the finite space.

When a subject pours urine as the test sample 50 over the electrochemical sensor configured in this way, the urine is supplied to the holding structure 30. Specifically, the urine is supplied into the cylinder of the cap portion 13 (that is, into the non-sealed finite space), using the opening 13a on one end side of the cap portion 13 as a supply port and the opening 13c on the other end side as a discharge port. At this time, due to the presence of the opening 13c as a discharge port, the urine is smoothly supplied into the finite space. Then, since the supplied urine is liquid, surface tension acts on it. Accordingly, a certain amount of urine is held in the non-sealed finite space that constitutes the holding structure 30.

That is, also in the electrochemical sensor according to another aspect of the present disclosure, in the same way as one aspect of the disclosure described above, when urine is poured over the electrochemical sensor, the holding structure 30 holds the urine to be brought into contact with the sensor electrodes 20. Thereby, the finite space located on the periphery of the sensor electrodes 20 is filled with the urine, and the immersion state of the sensor electrodes 20 in the filled urine is maintained. Therefore, the same effect as in the above-described one aspect of the present disclosure is obtained.

Incidentally, when the test sample 50 is urine, the urine supplied to the holding structure 30 may contain air bubbles. Since the holding structure 30 in the present disclosure has a configuration for holding the test sample 50 in the non-sealed finite space, it also has a feature that air bubbles can easily escape into the atmosphere. Further, for the purpose of facilitating removal of air bubbles contained in the urine, which is the test sample 50, the cap portion 13 that constitutes the holding structure 30 may include a hole or groove, etc., for removing the air bubbles. That is, the holding structure 30 may include an air bubble remover such as a hole or groove for removing the air bubbles contained in the test sample 50. When the air bubble remover is attached, by eliminating the air bubbles contained in the test sample 50, good inspection accuracy can be ensured in detecting the specific substance in the test sample 50.

Further, the urine supplied to the holding structure 30 may contain contaminants. Therefore, for example, the opening 13b serving as the supply port in the cap portion 13 may include a filter portion or a similar device for preventing the contaminants from entering the finite space. That is, the holding structure may be attached with a filter portion for preventing inflow of the contaminants of the test sample 50. When the filter portion is attached, it is possible to ensure good inspection accuracy in detecting the specific substance in the test sample 50 by removing the contaminants contained in the test sample 50.

Further, a groove, a guide member, etc., for guiding the urine, which is the test sample 50, to the opening 13b may be provided in the vicinity of the opening 13b serving as the supply port in the cap portion 13. That is, the holding structure 30 may be attached with an introduction structure such as a groove or a guide member that guides the test sample 50 to the holding structure 30, the test sample 50 being held by the holding structure 30. When the introduction structure is attached, the test sample 50 can be easily and reliably guided into the finite space, and the test sample 50 can be reliably held by the holding structure 30. Particularly, when the introduction structure is configured to utilize capillarity, it becomes possible to introduce the test sample 50 regardless of the posture (regardless of any posture) in use of the electrochemical sensor (particularly support 10), and therefore it becomes possible to guide the test sample 50, which improves convenience for a user.

### Modified Example

The embodiments of the present disclosure have been specifically described above. However, the present disclosure is not limited to each aspect described above, and various modifications can be made without departing from the scope of the present disclosure.

In each of the above aspects, explanation is given for an example in which the liquid test sample is urine collected from a subject, but the present disclosure is not limited to such aspects. For example, the liquid test sample may be body fluids such as blood, saliva, runny nose, sweat, tears, etc., in addition to urine. Further, liquid test samples are not limited to those derived from humans, and may be derived from animals such as dogs and cats.

Further, in each of the above aspects, explanation is given for an example in which the specific substance contained in the test sample is uric acid, but the present disclosure is not limited to such aspects. For example, the specific substance contained in the test sample may be uric acid, urinary sugar, arginine, albumin, etc.

In each of the above-described aspects, explanation is given for an example in which the urine is supplied by pouring urine from the subject, but the present disclosure is not limited thereto, and for example, even when the test sample is immersed in a container, etc., containing the test sample, it is possible to detect the specific substance contained in the test sample in exactly the same way.

In each of the above-described aspects, explanation is given for an example in which the concentration of the specific component in the test sample is measured by the three-electrode method, but the present disclosure is not limited to such an aspect. For example, the concentration of the specific substance in the test sample can be measured by a two-electrode method. In this case, the sensor electrodes may have two electrodes of a working electrode and a counter electrode (or reference electrode).

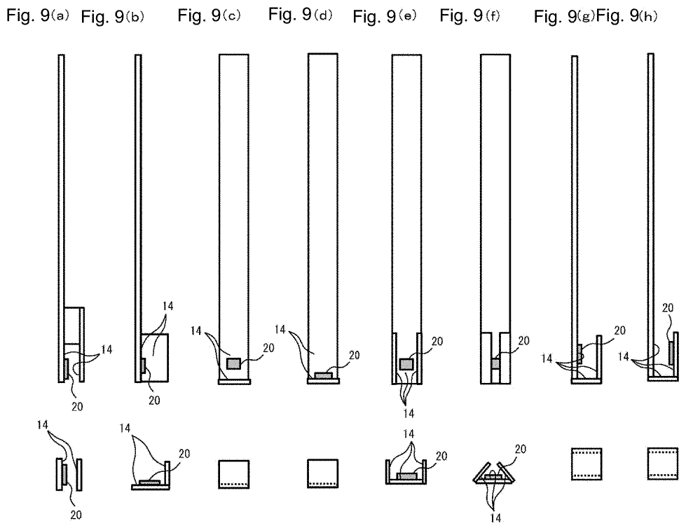

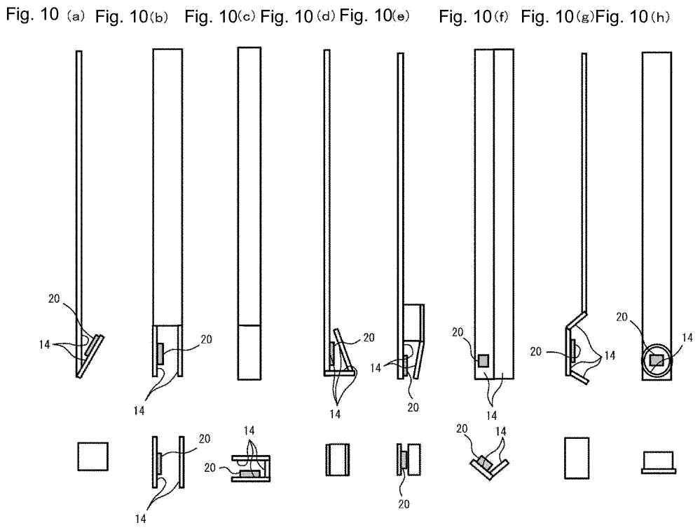

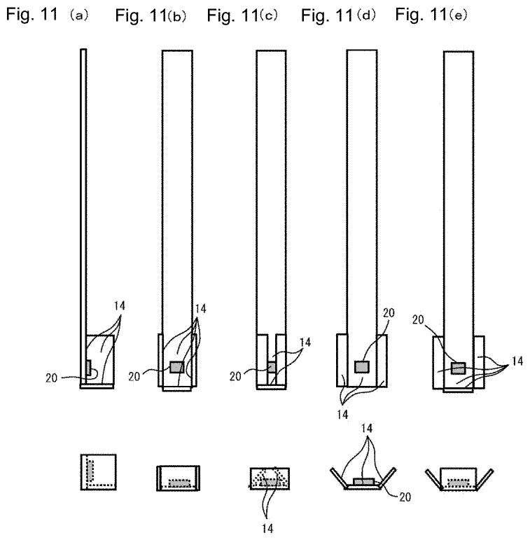

In one aspect of the present disclosure described above, explanation is given for an example in which the holding structure 30 has two constituent surfaces 11a and 12a in a substantially V-shaped positional relationship, and holds the test sample 50 in the non-sealed finite space formed by these. However, the present disclosure is not limited to such an aspect. For example, as illustrated in any of FIGS. 9(a) to (h), FIGS. 10(a) to (b), or FIGS. 11(a) to (e), an arrangement of each constituent surface 14 is not particularly limited, as long as the holding structure 30 is configured to have at least two (including three or more) constituent surfaces 14, thereby defining a non-sealed finite space between these configuration surfaces 14, so that the test sample 50 is in contact with the sensor electrodes 20 by holding the test sample 50 in the finite space.

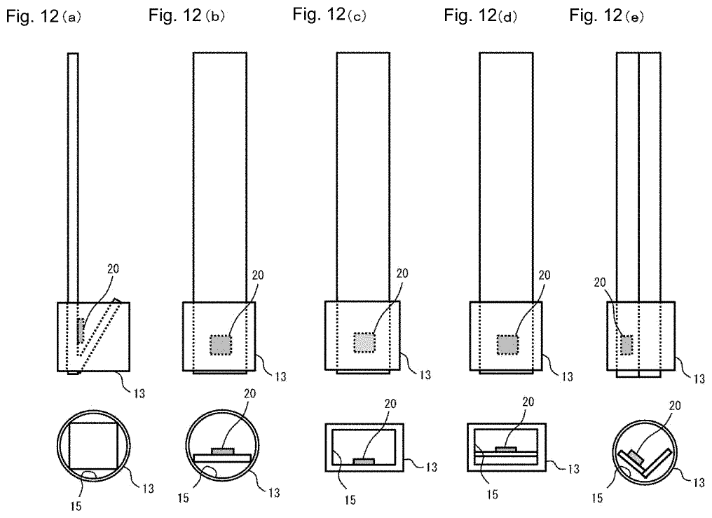

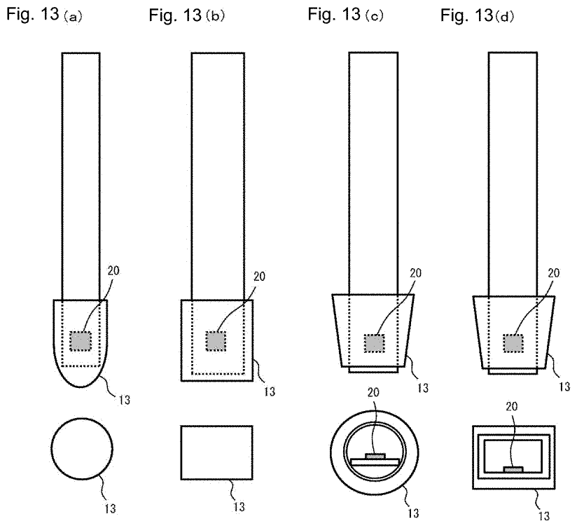

In other aspect of the present disclosure described above, explanation is given for an example in which the holding structure 30 has a wall surface formed to surround the sensor electrodes 20 and holds the test sample 50 in the finite space within the wall surface. However, the present disclosure is not limited to such an aspect. For example, as illustrated in either FIGS. 12(a) to (e) or FIGS. 13(a) to (d), an arrangement of the wall surface 15, a shape of the cap portion 13 having the wall surface 15, etc., are not particularly limited, as long as the holding structure 30 is configured to have a wall surface 15 formed to surround the sensor electrodes 20 so that the test sample 40 is in contact with the sensor electrodes 20 by holding the test sample 50 in the finite space within the wall surface 15.

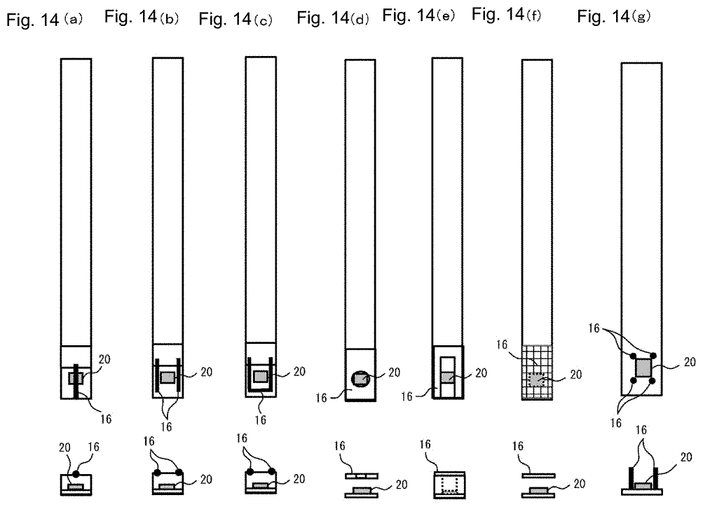

In each aspect of the present disclosure described above, explanation is given for an example of the non-sealed finite space defined by at least two constituent surfaces or walls surrounding the sensor electrodes. However, the present disclosure is not limited to such an aspect. For example, as illustrated in any of FIGS. 14(a) to (g), the holding structure 30 may have an attachment member 16 such as a net material, a bar material, a protruding structure, or a net-like structure arranged in the vicinity of the sensor electrodes 20 so that the test sample 50 is held in the non-sealed finite space configured between the sensor electrodes 20 and the attached member 16. In such a case, the arrangement, shape, etc., of the attachment member 16 are not particularly limited as long as the liquid test sample 50 can be held utilizing the surface tension of the test sample 50.

### Preferable Aspects of the Present Disclosure

Preferable aspects of the present disclosure will be supplementarily described below.

### (Supplementary Description 1)

According to an aspect of the present disclosure, there is provided an electrochemical sensor that electrochemically detects a specific substance in a liquid test sample by bringing it into contact with sensor electrodes arranged on a support, the electrochemical sensor including:

a holding structure that constitutes a non-sealed finite space facing the sensor electrodes and holds the test sample that is brought into contact with the sensor electrodes in the finite space.

### (Supplementary Description 2)

According to another aspect of the present disclosure, there is provided an electrochemical sensor, that electrochemically detects a specific substance in a liquid test sample by bringing it into contact with sensor electrodes arranged on a support, the electrochemical sensor including:

a holding structure that holds the test sample around the sensor electrodes so that only the test sample comes in contact with exposed surfaces of the sensor electrodes.

### (Supplementary Description 3)

According to further another aspect of the present disclosure, there is provided an electrochemical sensor that electrochemically detects a specific substance in a liquid test sample by bringing it into contact with sensor electrodes arranged on a support,

wherein a holding structure that holds the test sample while the test sample is in contact with the sensor electrodes, is provided on the support, and

a difference between intensity B of a detection signal obtained by immersing the sensor electrodes in the test sample stored in a container and intensity A of a detection signal obtained by the sensor electrodes while the holding structure holds the test sample, is 10% or less of the intensity A.

### (Supplementary Description 4)

Preferably, there is provided the electrochemical sensor according to any one of supplementary descriptions 1 to 3, wherein the holding structure is configured to hold the test sample utilizing a surface tension of the test sample.

### (Supplementary Description 5)

Preferably, there is provided the electrochemical sensor according to supplementary description 1, wherein the finite space in the holding structure is formed in a size and a shape to hold the test sample utilizing a surface tension of the test sample.

### (Supplementary Description 6)

Preferably, there is provided the electrochemical sensor according to any one of supplementary descriptions 1 to 5, wherein the holding structure has at least two constituent surfaces and is configured to hold the test sample between the two constituent surfaces.

### (Supplementary Description 7)

Preferably, there is provided the electrochemical sensor according to supplementary description 6, wherein the two constituent surfaces are arranged to face each other.

### (Supplementary Description 8)

Preferably, there is provided the electrochemical sensor according to any one of supplementary descriptions 1 to 5, wherein the holding structure has a wall surface formed to surround the sensor electrodes, and is configured to hold the test sample in a space within the wall surface.

### (Supplementary Description 9)

Preferably, there is provided the electrochemical sensor according to any one of supplementary descriptions 1 to 5, wherein the holding structure has an attachment member arranged in the vicinity of the sensor electrodes, and is configured to hold the test sample between the sensor electrodes and the attachment member.

### (Supplementary Description 10)

Preferably, there is provided the electrochemical sensor according to any one of supplementary descriptions 1 to 9, wherein the holding structure is configured such that a holding capacity of the test sample is 0.01 ml or more and 0.30 ml or less.

### (Supplementary Description 11)

Preferably, there is provided the electrochemical sensor according to any one of supplementary descriptions 1 to 10, wherein the holding structure is configured such that a contact angle of the test member when the test sample is brought into contact, is 0° or more and less than 90°.

### (Supplementary Description 12)

Preferably, there is provided the electrochemical sensor according to any one of supplementary descriptions 1 to 11, wherein the holding structure is configured to maintain a holding state of the test sample for at least 1 minute when no external force is applied.

### (Supplementary Description 13)

Preferably, there is provided the electrochemical sensor according to any one of supplementary descriptions 1 to 12, wherein the holding structure is configured such that there is no variation in the holding capacity of the test sample, or a variation in the holding capacity that occurs is less than an average value±10%, even when there is a change in a posture of the support or a change in an environmental condition within a predetermined range.

### (Supplementary Description 14)

Preferably, there is provided the electrochemical sensor according to any one of supplementary descriptions 1 to 13, wherein the holding structure is configured to detach the test sample in a held state from the holding structure by applying an external force.

### (Supplementary Description 15)

Preferably, there is provided the electrochemical sensor according to any one of supplementary descriptions 1 to 14, wherein the holding structure is configured to be detachable from the support.

### (Supplementary Description 16)

Preferably, there is provided the electrochemical sensor according to any one of supplementary descriptions 1 to 15, wherein the holding structure is attached with an introduction structure that guides the test sample to the holding structure, the test sample being held by the holding structure.

### (Supplementary Description 17)

Preferably, there is provided the electrochemical sensor according to any one of supplementary descriptions 1 to 16, wherein the holding structure is attached with a filter portion for preventing inflow of contaminants mixed in the test sample.

### (Supplementary Description 18)

Preferably, there is provided the electrochemical sensor according to any one of supplementary descriptions 1 to 17, wherein the holding structure is attached with a bubble remover that removes bubbles contained in the test sample.

### (Supplementary Description 19)

Preferably, there is provided the electrochemical sensor according to any one of supplementary descriptions 1 to 18, wherein the holding structure also has a protective function to prevent an object other than the test sample from coming into contact with the sensor electrodes.

### (Supplementary Description 20)

Preferably, there is provided the electrochemical sensor according to any one of supplementary descriptions 1 to 19, wherein the holding structure also has an evaporation prevention function for preventing evaporation of the test sample in a held state.

### (Supplementary Description 21)

Preferably, there is provided the electrochemical sensor according to any one of supplementary descriptions 1 to 20, wherein the sensor electrodes has a working electrode and a counter electrode, and the working electrode comprises a diamond film that causes an oxidation-reduction reaction on a surface when a predetermined voltage is applied with the test sample present between the working electrode and the counter electrode.

### (Supplementary Description 22)

Preferably, there is provided the electrochemical sensor according to any one of supplementary descriptions 1 to 21, wherein the holding structure is configured to hold the test sample in such a manner that at least an entire exposed surface of the working electrode is in contact with the test sample.

### (Supplementary Description 23)

Preferably, there is provided the electrochemical sensor according to supplementary descriptions 21 or 22, wherein the holding structure is configured to hold the test sample in a region having an area larger than that of the working electrode.

### (Supplementary Description 24)

Preferably, there is provided the electrochemical sensor according to any one of supplementary descriptions 21 to 23, wherein the holding structure is configured to hold the test sample in such a manner of maintaining a thickness equal to or greater than a diffusion length of a component to be detected with respect to the exposed surface of the working electrode.

### (Supplementary Description 25)

Preferably, there is provided the electrochemical sensor according to any one of supplementary descriptions 1 to 24, wherein the holding structure comprises a light-transmitting material.

### (Supplementary Description 26)

Preferably, there is provided the electrochemical sensor according to any one of supplementary descriptions 1 to 25, wherein the holding structure comprises a material that does not impregnate the test sample.

### (Supplementary Description 27)

Preferably, there is provided the electrochemical sensor according to any one of supplementary descriptions 1 to 26, wherein the holding structure comprises an insulating material.

### (Supplementary Description 28)

Preferably, there is provided the electrochemical sensor according to any one of supplementary descriptions 1 to 27, wherein the holding structure comprises a flexible material.

### (Supplementary Description 29)

Preferably, there is provided the electrochemical sensor according to any one of supplementary descriptions 1 to 28, wherein the holding structure comprises a material that does not contain a component that elutes into the test sample.

### (Supplementary Description 30)

Preferably, there is provided the electrochemical sensor according to any one of supplementary descriptions 1 to 29, wherein the holding structure comprises a hydrophilic material.

### (Supplementary Description 31)

Preferably, there is provided the electrochemical sensor according to any one of supplementary descriptions 1 to 30, wherein the holding structure comprises the same material as the support.

### (Supplementary Description 32)

Preferably, there is provided the electrochemical sensor according to any one of supplementary descriptions 1 to 30, wherein the holding structure comprises a material different from that of the support.

### (Supplementary Description 33)

Preferably, there is provided the electrochemical sensor according to any one of supplementary descriptions 1 to 32, wherein the holding structure comprises a material having a smooth surface at a contact portion with the test sample.

Preferably, there is provided the electrochemical sensor according to any one of supplementary descriptions 1 to 32, wherein the holding structure comprises a material having a rough surface at a contact portion with the test sample.

### (Supplementary Description 35)

Preferably, there is provided the electrochemical sensor according to any one of supplementary descriptions 1 to 34, wherein the holding structure comprises polyethylene or epoxy resin.

### (Supplementary Description 37)

Preferably, there is provided the electrochemical sensor according to any one of supplementary descriptions 1 to 35, wherein the test sample is human or animal urine or body fluid.

### DESCRIPTION OF SIGNS AND NUMERALS

10 Support
11 Base piece portion
11a Constituent surface
12 Protruding piece portion
12a Constituent surface
13 Cap portion
13a Constituent surface (wall surface)
13b Opening
13c Opening
14 Constituent surface
15 Wall surface
16 Attachment member
20 Sensor electrodes
21 Working electrode
22 Counter electrode
23 Reference electrode
30 Holding structure
50 Test sample

1. An electrochemical sensor that electrochemically detects a specific substance in a liquid test sample by bringing it into contact with sensor electrodes arranged on a support, the electrochemical sensor including:
a holding structure that constitutes a non-sealed finite space facing the sensor electrodes and holds the test sample that is brought into contact with the sensor electrodes in the finite space.

2. The electrochemical sensor according to claim 1, wherein the holding structure is configured to hold the test sample utilizing a surface tension of the test sample.

3. The electrochemical sensor according to claim 1, wherein the holding structure has at least two constituent surfaces and is configured to hold the test sample between the two constituent surfaces.

4. The electrochemical sensor according to claim 1, wherein the holding structure is configured such that a holding capacity of the test sample is 0.01 ml or more and 0.30 ml or less.

5. The electrochemical sensor according to claim 1, wherein the holding structure is configured such that there is no variation in the holding capacity of the test sample, or a variation in the holding capacity that occurs is less than an average value±10%, even when there is a change in a posture of the support or a change in an environmental condition within a predetermined range.

6. The electrochemical sensor according to claim 1, wherein the holding structure is configured to detach the test sample in a held state from the holding structure by applying an external force.

7. The electrochemical sensor according to claim 1, wherein the holding structure also has a protective function to prevent an object other than the test sample from coming into contact with the sensor electrodes.

8. The electrochemical sensor according to claim 1, wherein the sensor electrodes have a working electrode and a counter electrode, and the working electrode comprises a diamond film that causes an oxidation-reduction reaction on a surface when a predetermined voltage is applied with the test sample present between the working electrode and the counter electrode.

9. The electrochemical sensor according to claim 8, wherein the holding structure is configured to hold the test sample in such a manner that at least an entire exposed surface of the working electrode is in contact with the test sample.

10. The electrochemical sensor according to claim 1, wherein the holding structure comprises a light-transmitting material.

11. The electrochemical sensor according to claim 1, wherein the holding structure comprises an insulating material.

12. The electrochemical sensor according to claim 1, wherein the test sample is human or animal urine or body fluid.

13. The electrochemical sensor according to claim 2, wherein the holding structure has at least two constituent surfaces and is configured to hold the test sample between the two constituent surfaces.

14. The electrochemical sensor according to claim 2, wherein the holding structure is configured such that a holding capacity of the test sample is 0.01 ml or more and 0.30 ml or less.

15. The electrochemical sensor according to claim 3, wherein the holding structure is configured such that a holding capacity of the test sample is 0.01 ml or more and 0.30 ml or less.

16. The electrochemical sensor according to claim 4, wherein the holding structure is configured such that there is no variation in the holding capacity of the test sample, or a variation in the holding capacity that occurs is less than an average value±10%, even when there is a change in a posture of the support or a change in an environmental condition within a predetermined range.

17. The electrochemical sensor according to claim 2, wherein the holding structure is configured to detach the test sample in a held state from the holding structure by applying an external force.

18. The electrochemical sensor according to claim 3, wherein the holding structure is configured to detach the test sample in a held state from the holding structure by applying an external force.

19. The electrochemical sensor according to claim 4, wherein the holding structure is configured to detach the test sample in a held state from the holding structure by applying an external force.

20. The electrochemical sensor according to claim 5, wherein the holding structure is configured to detach the test sample in a held state from the holding structure by applying an external force.
